(19) United States
(12) Patent Application Publication
(10) Pub. No.: US 2018/0008478 A1
(43) Pub. Date: Jan. 11, 2018

(54) DIAPERS WITH URINE DETECTION FUNCTIONALITY AND METHODS FOR DETECTING TRACE ELEMENTS

(71) Applicant: Baby Care Development Technologies Corporation, City of Industry, CA (US)

(72) Inventor: Jie Xu, Harbin (CN)

(21) Appl. No.: 15/643,340

(22) Filed: Jul. 6, 2017

(30) Foreign Application Priority Data
Jul. 6, 2016 (CN) 201610528043.6

(51) Int. Cl. A61F 13/42; A61F 13/539; A61B 5/00; A61B 5/01; A61F 13/51; A61F 13/15

(52) U.S. Cl. CPC A61F 13/42; A61B 5/01; A61F 13/51; A61F 13/539; A61B 5/002; A61F 2013/15146; A61F 2013/423; A61F 2013/424; A61F 2013/427; A61F 2013/53908; A61F 2013/421

(57) ABSTRACT

A diaper with urine detection functionalities is provided. The diaper includes a urine isolation layer, an absorbent core layer, and an outer layer. A urine detection device is disposed between the urine isolation layer and the absorbent core layer. The isolation layer has an open end that is embedded in a sleeve of the absorbent core layer. The urine detection device comprises a detection circuit and a test paper composite. The test paper composite includes: a trace element test paper, an antibiotic test paper, and a urine routine test paper. Each test paper is made up of a plurality of test strips for detecting different elements. Each test strip produces results according to a different color indicator scheme.

### CROSS REFERENCE TO RELATED APPLICATIONS

This application claims the benefit and priority of Chinese Patent Application No. 201610528043.6, filed on Jul. 6, 2016, which is incorporated herein by reference in its entirety.

### TECHNICAL FIELD

The invention relates to a diaper with urine detection function and a urine detection method.

### BACKGROUND

At present, the widespread use of baby diapers instead of traditional diapers has the advantages of convenience, but parents need to regularly check whether one needs to replace diapers, because when the replacement is not timely, soaked and/or soiled diapers will cause the burns and rashes on the skin of the baby. Improved diapers with urine display function, such as a diaper wet color suitable for hot melt adhesive for disposable sanitary products using methods such as those disclosed in Chinese Patent Application No. 201310469895.9, but with significant application usually diapers urine chemical principle, only after use of color to remind, parents still need regular observation. It is not very convenient for use at night.

A urine test research more than a thousand children of Jiangsu, Zhejiang, Shanghai from Fudan University, shows that 58% of children were detected with at least one kind of antibiotics, ¼ detected with more than 2 kinds of antibiotics, some samples even have 6 kinds of antibiotics, some antibiotics are commonly used antibiotics in animal husbandry. WHO (World Health Organization) has released that antibiotic resistant bacteria are spreading all over the world. (April 17th, reference news). Antibiotic resistance, like bad smog, will evolve into an environmental crisis that threatens everyone. Inappropriate use of drugs causes and intensifies changes in bacteria and other microorganisms, and eventually leads to ineffective use of drugs for the treatment of infections, which is the source of antibiotic resistance. The widespread use of antibiotics in animal husbandry can lead to the development of antibiotic resistance. Antibiotic resistance is spread in livestock and spreads through food borne diseases and other infections. A common example is the detection of commonly used antibiotics in children's urine. In the human body, the use of inappropriate drugs can also cause antibiotic resistance. There is no convenient way to test the antibiotic levels in children's urine.

In addition, trace elements are necessary for children during their growth and development. The trace elements in urine can be used to infer the content of trace elements in children, and then to analyze the nutritional status of children.

The existing diaper cannot detect excrement such as urine, thereby analyzing the health status of users, etc., and further improvement is needed.

### SUMMARY

The technical problem to be solved by this disclosure is to provide diapers (e.g., diapers for babies, adult diapers, etc.), with urine testing functions, including body temperature, microelements, antibiotics, and routine urine testing, and access to consumer health information.

In order to solve the technical problem, the disclosure provides diapers with urine detection functions. In some embodiments, the diaper comprises, from the interior to the exterior, a urine isolation layer, an absorbent core layer and an outer layer. A urine detection device may be disposed in between the urine isolation layer and the absorbent core layer. The urine isolation layer has an open end embedded in a sleeve of the absorbent core layer. The urine detection device may comprise a detection circuit and a test paper composite. The test paper composite includes: a trace element test paper, an antibiotic test paper and a urine routine test paper. Each test paper is made up of test strips for detecting different elements. Each test strip produces results according to a different color indicator scheme.

In some embodiments, the detection circuit of the diaper with urine detection function comprises: a controller, a temperature sensor communicatively coupled to the controller and configured to measure a temperature of a user, a humidity sensor communicatively coupled to the controller and configured to detect a humidity of the absorbent core layer, a wireless transmission module communicatively coupled to the controller and configured to communicate wirelessly with a remote mobile terminal, and a power supply module configured to provide power to the controller, the temperature sensor, the humidity sensor, and the wireless transmission module.

In some embodiments, the wireless transmission module may perform wireless communication according to the Bluetooth protocol or the WI-FI protocol.

In some embodiments, the test paper composite comprises a trace elements detection test paper for detecting trace elements, an antibiotic detection test paper for detecting antibiotics, and a urine test strip for detecting urine.

In some embodiments, each of the types of test paper includes one or more different test paper components for detecting different types of substances, each test component provides test results according to a different color indication scheme.

In some embodiments, each test component may have a length of 5-30 mm, a width of 5-20 mm, and a thickness of 1-5 mm.

In some embodiments, the urine detection test paper (urine test strip) performs at least one of the following testing or detecting: a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

In some embodiments, the trace elements detection testing of the diapers according to some embodiments of the disclosure includes: calcium detection test, test of iron and zinc elements test.

In some embodiments, the calcium testing performed in the diapers according to some embodiments of the disclosure includes excessive calcium test and test for calcium deficiency. The excess calcium test provides an indicator in a blue color when the calcium content is normal, and provides an indicator in a purple color when excessive calcium is detected. The test for calcium deficiency provides an indicator in a blue color when calcium a calcium deficiency is detected, and provides an indicator in a purple-red color when the calcium content is normal.

In some embodiments, test paper component corresponding to the calcium test comprises porous nano materials, wherein the porosity of the porous nano materials is 40%-80%, detection test materials are disposed on the porous nano material.

In some embodiments, the zinc test paper may be manufactured by soaking filter paper in a solution, drying the filter paper, and trimming the filter paper, the solution contains acid, a mixture of sodium thiocyanate and crystal violet, the acid may include hydrochloric acid or sulfuric acid.

In some embodiments, a zinc detection color card is provided. The zinc detection color card is generated by adding different content of zinc on the zinc detection test paper and then determine a changed color on the zinc detection test paper.

The disclosure also provides a method for detecting trace elements, a urine testing device is disposed between the urine isolation layer and the absorbent core layer of a diaper, the urine test device is communicatively coupled with a mobile terminal using wireless connections, the testing steps including.
(1) the mobile terminal scans the two-dimensional code on the diaper to perform a pairing process
(2) monitor humidity in real time to determine if diaper is wet, if it is the next step;
(3) the mobile terminal prompts to change the diaper, and the trace element in the urine detection device detects the different color blocks displayed after the urine sample is soaked in urine, and judges whether there is abnormal trace element.

Preferably, the method of detection of trace elements according to various embodiments of the disclosure, step (1) and step (2) also includes a real-time detection device for detecting the temperature of urine diapers, mobile terminal display temperature and set temperature alarm value, when the temperature anomaly of mobile terminal alarm.

Further, the method of detection of trace elements according to various embodiments of the disclosure, step (3) according to the different color to determine trace elements in the urine test paper for the detection device in the detection shows whether there is abnormal trace element, which comprises the following steps:
(a) Uncover the urine layer and take the test paper with the moving end;
(b) Test paper image recognition, block and standard color value of each block;
(c) Determine whether the test paper is abnormal, if it is the mobile terminal alarm prompt.

The diapers according to various embodiments of the disclosure provide simple structure, low cost, can have a urine detection function, including temperature, trace elements, antibiotics, and routine urine test, so as to obtain users (children) of the health information, the mobile terminal can be combined with the use of long-term health monitoring of children.

### BRIEF DESCRIPTION OF THE DRAWINGS

FIG. 1 illustrates a diaper with the urine detection function according to some embodiments of the disclosure;

FIG. 2 is a schematic diagram of a circuit for the urine detection device according to some embodiments of the disclosure;

FIG. 3 illustrates a diaper with a urine detection function having a code according to some embodiments of the disclosure;

FIG. 4 is a schematic diagram of a urine detection device when the urine isolation layer is removed from the diaper according to some embodiments of the disclosure; and

FIG. 5 is a flow chart of a urine detection method according to some embodiments of the disclosure.

Embodiments of the present disclosure and their advantages may be understood by referring to the detailed description herein. It should be appreciated that reference numerals may be used to illustrate various elements and features provided in the figures. The figures may illustrate various examples for purposes of illustration and explanation related to the embodiments of the present disclosure and not for purposes of any limitation.

### DETAILED DESCRIPTION

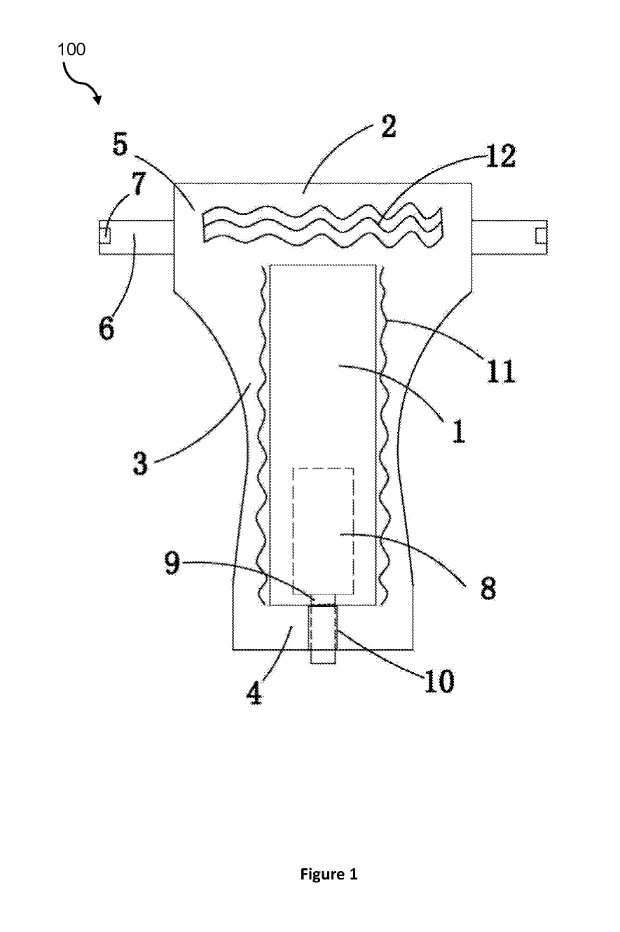

As shown in FIG. 1, the disclosure provides a diaper 100 (e.g., a baby diaper, an adult diaper, etc.) with urine detection functions. The diaper 100 includes absorbent core layer 1, rear waist portion 2, crotch portion 3, front waist portion 4; wing portion 5, adhesive tape 6, adhesive buckle 7, test paper composite 8, detection circuit 9, open end card sleeve 10 of the urine isolation layer, leakage prevention edge 11, and elastic band 12.

The diaper 100 includes, from interior to exterior, urine isolation layer, absorbent core layer 1, and the outer layer of the pants. The outer layer of the pants comprises rear waist portion 2, crotch portion 3, and front waist portion 4.

The two sides of the rear waist portion 2 include wing portion 5 and one end of adhesive tape 6. The other end of the adhesive tape 6 is a free end which is provided with an adhesive buckle 7. The urine isolating layer and the absorbent core layer 1 is provided with a urine detection device. The urine detection device comprises a detection circuit 9 and test paper composite 8. The urine isolation layer is provided with an open end that is embedded in the clamping sleeve 10 arranged in the absorption core layer 1. Inside the outer pants body is provided with a two contraction of 11 two flange leak, leak proof contraction of the flange 11 is located in the absorption core layer 1 on both sides, makes it keep in the baby liquid absorption core layer 1, no leakage from both sides. The inner side of the back waist 2 is transversely provided with an elastic contraction band 12, and both ends of the elastic contraction band 12 are fixedly connected with the two sides of the inside of the 2 sides of the back waist to prevent leakage of the toilet liquid.

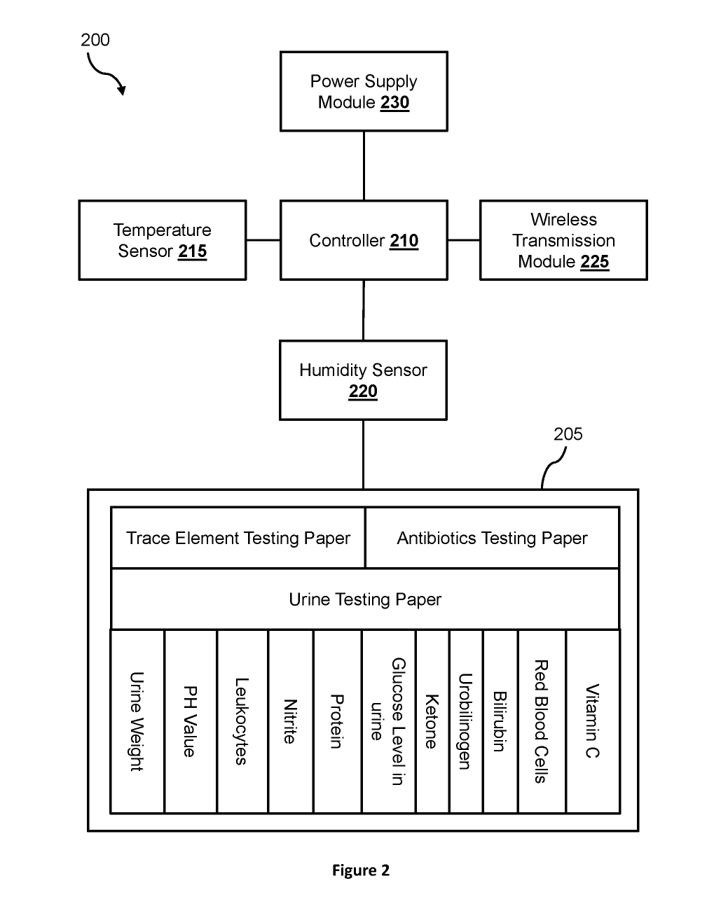

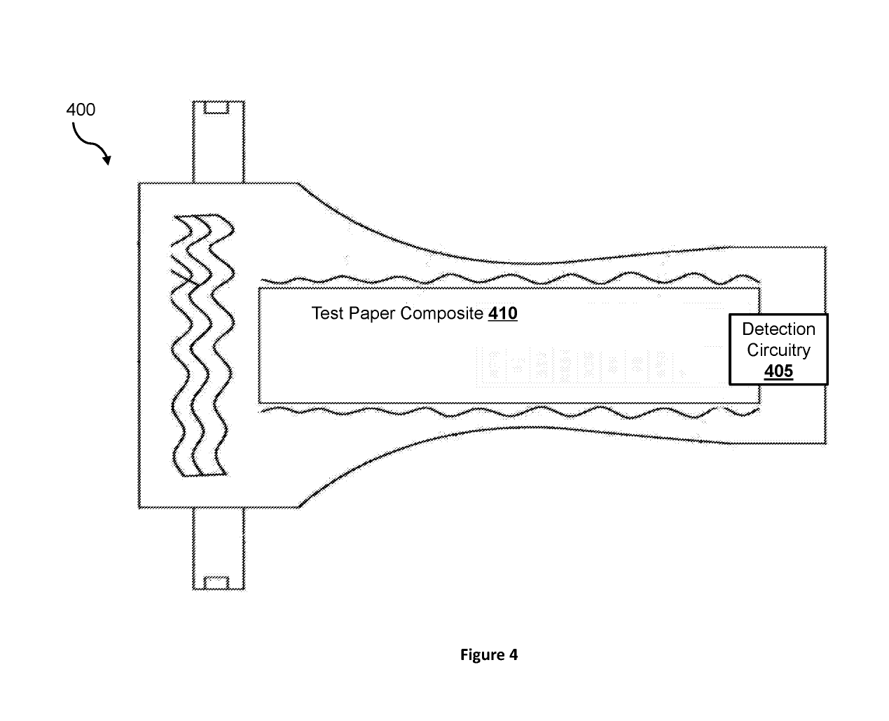

As shown in FIG. 2 and FIG. 4, the disclosed diaper includes a detection circuit. FIG. 2 illustrates an example detection circuit 200 and a test paper composite 205. the detection circuit of the diaper with urine detection function comprises: a controller 210, a temperature sensor 215 communicatively coupled to the controller and configured to measure a temperature of a user, a humidity sensor 220 communicatively coupled to the controller and configured to detect a humidity of the absorbent core layer, a wireless transmission module 225 communicatively coupled to the controller and configured to communicate wirelessly with a remote mobile terminal, and a power supply module 230 configured to provide power to the controller 210, the temperature sensor 215, the humidity sensor 220, and the wireless transmission module 225. In some embodiments, the wireless transmission module 225 may perform wireless communication according to the Bluetooth protocol or the WI-FI protocol.

FIG. 4 illustrates an example diaper 400 with a detection circuitry 405 and a test paper composite 410. In some embodiments, the test paper composite 410 is the same as test paper composite 205 of FIG. 2.

The disclosure discloses a diaper with the function of urine detection, wherein the test paper composite comprises a trace element test paper, an antibiotic detection paper and a urine routine test paper. Each of the trace element test paper, the antibiotic detection paper and the urine routine test paper may be made up of a plurality of test paper components for detecting different substances. Each of the plurality of test paper components may provide a test result according to a different color indication scheme. The test paper component for each test item may have a length of 5-30 mm, a width of 5-20 mm, and a thickness of 1-5 mm.

The urine detection test paper (urine test strip) performs at least one of the following testing or detecting: a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

The trace element test comprises: a calcium element test paper, an iron element test paper and a zinc element test paper.

The test paper of the calcium element may comprise a porous nanometer material, and the porosity of the porous nanometer material is 40%-80%, and the detecting reagent is adhered to the porous nanometer material. Test for calcium includes a test for excessive calcium and a test for calcium deficiency. The test for excessive calcium may provide an indicator of a blue color when normal calcium content is detected and may provide an indicator of a purple-red color when excessive calcium content is detected. The test for calcium deficiency may provide an indicator of a blue color when calcium deficiency is detected and may provide an indicator in a purple/red color when normal calcium content is detected.

The preparation method of the calcium element test paper comprises the following steps:
(1) Preparation of impregnating solution: 3.5 g-4.5 g buffer, 50 g-100 g surfactant, 2.5 g-5.5 g masking agent, 0.01 g-1.5 g calcium indicator and 0.9 g-1.5 g EDTA are added to volumetric flask, and the volume is fixed to 1000 ml;

Wherein, the buffer is prepared with sodium hydroxide, and the surface active agent is a combination of polyvinylpyrrolidone and polyethylene glycol, and the masking agent is a combination of sodium tartrate and 8-hydroxyquinoline;
(2) The porous nanometer material is soaked in the dipping liquid, soaked 20 min, taken out and dried in an oven, and the oven temperature is 30-50 DEG C.

The zinc detection test paper is prepared by dipping, drying and cutting the filter paper in the solution, and the solution comprises a mixture of strong acid, sodium thiocyanate and crystal violet, wherein the strong acid is hydrochloric acid or sulfuric acid.

The preparation method of the zinc element test paper 1:
(1) Preparation solution: adding 10-40 g, sulfuric acid, 5-30 g, sodium thiocyanate, 0.1-2 g crystal violet to volumetric flask, constant volume to 1000 ml;
(2) Immerse the filter paper completely in the solution, then soak it out and dry in the oven;
(3) The zinc ion in the dried urine is detected and the filter paper is cut into test paper strips.

The preparation method of the zinc element test paper 2:
(1) Preparation solution: adding 5-30 g hydrochloric acid, 5-30 g sodium thiocyanate, 0.1-2 g crystal violet to volumetric flask, and constant volume to 1000 ml;
(2) Immerse the filter paper completely in the solution, soak 10 min, then remove and dry in the oven;
(3) The zinc ion in the dried urine is detected and the filter paper is cut into test paper strips.

According to the color of the zinc element test paper added with different zinc content standard liquid, a new element detection standard color ratio card is made. Include the following steps:
(1) Prepare 0 g/L, 0.1 g/L, 0.15 g/L, 0.2 g/L, 0.5 g/L, 1 g/L, 1.1 g/L, 1.2 g/L, 1.3 g/L series of zinc containing standard liquid;
(2) The test paper prepared by embodiment 1 or embodiment 2 is dipped in the standard solution containing zinc ion, then removed and dried for 2 seconds, and the color is completely finished after 20 seconds;
(3) According to the paper's color preparation than color card, color card color is green, blue, blue and purple three order.

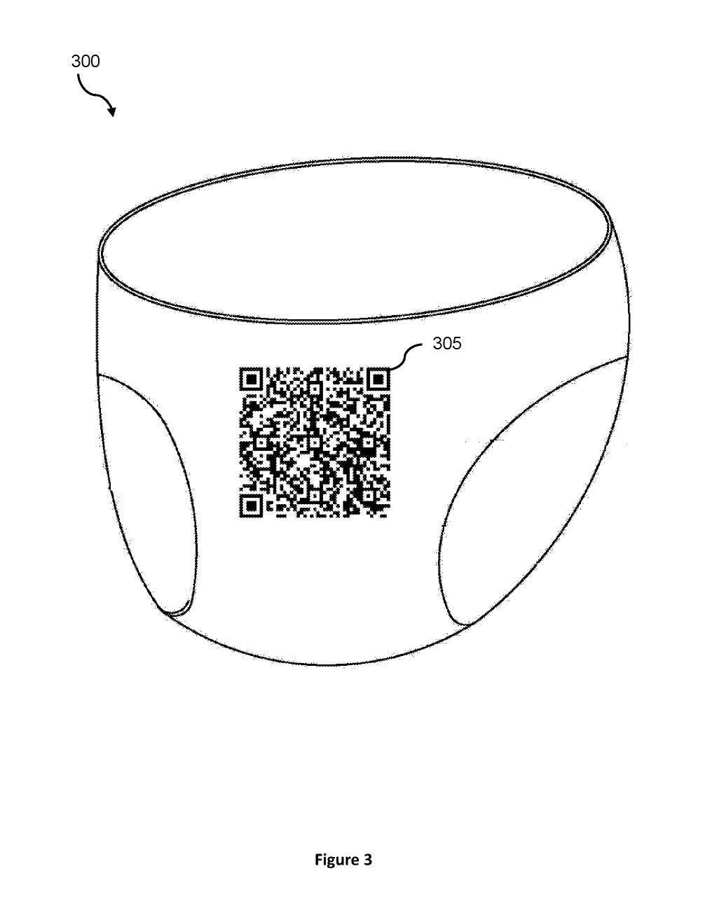

As shown in FIG. 3, an external sketch of a diaper 300 for children with urine detection function is provided. The diaper has a code 305 that can be scanned by a remote mobile terminal (not shown) for pairing between the remote mobile terminal and a urine detection device of the diaper 300.

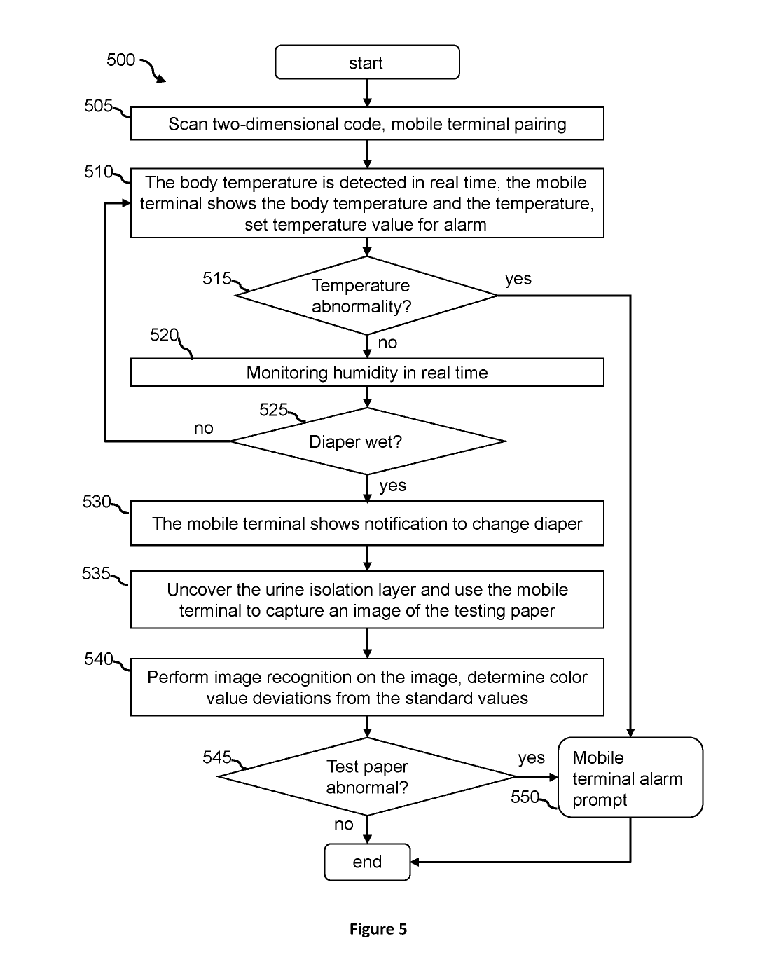

As shown in FIG. 5, the disclosure also provides a method for detection of trace elements, urine detection device is arranged between the isolation layer in the urine in children's diapers and absorption core layer, wherein the urine detection device is connected with the wireless mobile terminal, including the detection step:
(1) In block 505, the mobile terminal scans the two-dimensional code on the diaper (an example of the code is shown as code 305 in FIG. 3) for pairing;
(2) In block 510, the urine detection device of the diaper detects the body temperature in real time, the mobile terminal displays the body temperature, sets the alarm value of the temperature, and when the temperature is determined to be abnormal (in block 515), carries out the alarm prompt by the mobile terminal in block 550. If the temperature is not abnormal, go to block 520;
(3) In block 520, monitor humidity in real time to determine whether diaper has wet urine, if it is determined to be wet (in block 525), go to block 530. If diaper is not wet, return block 510;
(4) in block 530, the mobile terminal prompts the user to change the diaper, and according to the trace element in the urine detection device, the different color blocks displayed after the urine sample soaked in the test paper are judged whether the trace elements are abnormal. Further steps are included:
   (4.1) In block 535, uncover the urine isolation layer and take an image of the test paper composite with the mobile terminal (as shown in FIG. 4);
   (4.2) In block 540, perform image recognition on the test paper image, compare detected color with standard color value for each test paper component;
   (4.3) In block 545, determine whether the urine is abnormal according to the test paper results, if it is determined to be abnormal, the mobile terminal provides (in block 550) an alarm prompt.

The diapers according to various embodiments of the disclosure provide simple structure, low cost, can have a urine detection function, including temperature, trace elements, antibiotics, and routine urine test, so as to obtain users (children) of the health information, the mobile terminal can be combined with the use of long-term health monitoring of children.

The present disclosure, the accompanying figures, and the claims are not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, persons of ordinary skill in the art will recognize that changes may be made in form and detail without departing from the scope of the present disclosure.

1. A diaper comprising:
a urine isolation layer;
a urine absorbent core layer disposed on the urine isolation layer;
an outer layer disposed on the urine absorbent core layer, and
a urine detection device disposed in between the urine isolation layer and the urine absorbent core layer,
wherein the urine isolation layer comprises an opening at one end of the urine isolation layer, the opening embedded in a sleeve of the absorption core layer.

2. The diaper of claim 1, wherein the urine detection device comprises a detection circuit and a test paper composite.

3. The diaper of claim 2, wherein the detection circuit comprises:
a controller;
a temperature sensor communicatively coupled to the controller and configured to obtain a temperature of a user;
a humidity sensor communicatively coupled to the controller and configured to detect a humidity of the absorbent core layer;
a wireless transmission module communicatively coupled to the controller and configured to communicate signals wirelessly with a remote mobile terminal; and
a power supply module configured to provide power to the controller, the temperature sensor, the humidity sensor, and the wireless transmission module.

4. The diaper of claim 3, wherein the wireless transmission module is operable in a Bluetooth wireless transmission mode and/or a WI-FI wireless transmission mode.

5. The diaper of claim 2, the test paper composite comprises a trace elements detection test paper, an antibiotic detection test paper, and a urine test strip.

6. The diaper of claim 5, wherein each of the trace elements detection test paper, the antibiotic detection test paper, and urine test strips comprises a plurality of test paper components, wherein each of the plurality of test paper components provides test results according to a different color indication scheme.

7. The diaper of claim 6, wherein each of the plurality of test paper components has a length between 5-30 mm, a width between 5-20 mm, and a thickness between 1-5 mm.

8. The diaper of claim 6, wherein the test paper components of the urine test strip detects at least a weight of urine detected in the diaper, a PH value of the urine, leukocytes content in the urine, nitrite content in the urine, protein content in the urine, glucose content in the urine, ketone bodies content in the urine, urobilinogen content in the urine, bilirubin content in the urine, erythrocyte content in the urine, and vitamin C content in the urine.

9. The diaper of claim 6, wherein the trace elements detection test paper comprises: a calcium detection test component, an iron detection test component, and a zinc detection test component.

10. The diaper of claim 9, wherein the calcium detection test component comprises porous nanomaterials having a porosity between 40% and 80%, wherein calcium detection materials are disposed on the porous nanomaterials.

11. The diaper of claim 10, wherein the calcium detection test component comprises a subcomponent for detecting excessive calcium level and a subcomponent for detecting a lack of calcium, wherein a blue color in the subcomponent for detecting excessive calcium level indicates a normal calcium level, a red color in the subcomponent for detecting excessive calcium level indicates an excessive calcium level, a blue color in the subcomponent for detecting a lack of calcium indicates a lack of calcium, and a red color in the subcomponent for detecting a lack of calcium indicates a normal calcium level.

12. The diaper of claim 9, wherein the zinc detection test paper is manufactured by soaking a filter paper in a solution, drying and cutting the filter paper, wherein the solution comprises acid, sodium thiocyanate, and crystal violet, wherein the acid comprises hydrochloric acid or sulfuric acid.

13. As described in claim 12, wherein a standard color card for the zinc detection test component is generated by recording different color indications from the zinc detection test component based on additions of different content of zinc to the zinc detection test component.

14. A method for detecting trace elements in a diaper, comprising:
scanning, by a mobile terminal, a code on the diaper to perform a pairing between the mobile terminal and a detection device of the diaper;
monitoring, by the detection device, a humidity of the diaper based on a color change of a trace elements detection test paper of the diaper to determine if the diaper is wet; and
in response to a determination that the diaper is wet, providing, by the mobile terminal, an alert for changing diapers.

15. The method of claim 14, further comprising:
determining, by the detection device, a temperature of the diaper, wherein the mobile terminal stores a normal temperature range; and
in response to a determination that the temperature of the diaper is an abnormal temperature based on the stored normal temperature range, providing, by the mobile terminal, an alert.

16. The method of claim 15, wherein determining the humidity of the diaper comprises:
removing a urine isolation layer of the diaper and capturing, by the mobile terminal, an image of the trace elements detection test paper;
detecting, by the mobile terminal, a color indication of the trace elements detection test paper;
determining, by the mobile terminal, an abnormal condition based on a comparison between the detected color indication and a stored color standard; and
providing, by the mobile terminal, an alert in response to determining the abnormal condition.
